(19) United States
(12) Patent Application Publication
(10) Pub. No.: US 2025/0288761 A1
(43) Pub. Date: Sep. 18, 2025

(54) Device for Helping User to Fix Oxygen Hose

(71) Applicant: Jie PENG, Hunan (CN)

(72) Inventor: Jie PENG, Hunan (CN)

(21) Appl. No.: 18/607,713

(22) Filed: Mar. 18, 2024

(51) Int. Cl. A61M 16/00; A61M 16/06; A61M 16/08

(52) U.S. Cl. CPC A61M 16/0003; A61M 16/06; A61M 16/0875; A61M 2202/0208; A61M 2209/082

(57) ABSTRACT

A device for helping a user to fix an oxygen hose comprises a base, a bracket and a clamp, the base is detachably attached to a plane, the bracket is rotatably engaged on the base, at least one receiving part is arranged on the base; the bracket comprises a fixed end, a movable end and at least one rotating feature, the receiving part receives the fixed end and forms a rotatable connection with the fixed end; the clamp is arranged on the bracket far away from the fixed end; the rotating feature is located between the movable end and the fixed end and facilitates the rotating movement of the movable end around at least one rotating axis of the fixed end, the rotating feature comprises an at least partially spherical surface, thereby facilitating the controlled change of the position of the movable end relative to the user by the user.

### TECHNICAL FIELD

The present invention relates to the field of medical devices, in particular to a device for helping a user to fix an oxygen hose.

### BACKGROUND

For some people with sleep apnea syndrome, a ventilator must be used during sleep, which can help improve the breathing effect of patients during sleep; In addition, ventilators are also commonly used in clinical treatment, such as anesthesia breathing management and emergency resuscitation, but the hose of ventilators is difficult to fix, which will bring troubles to users and affect the use effect of ventilators.

The U.S. Pat. No. 854,196B1 discloses a breathing hose support system, in which a vertical support rod is arranged on a base, a rotating assembly including a spring is arranged on the vertical support rod, and a horizontal support rod extends from the rotating assembly, and the horizontal support rod is movably connected with the bearing assembly to support the breathing hose. The above combination can support the breathing hose and freely adjust the position of the breathing hose. However, the above solution still has some shortcomings.

In the above solution, because the combination of the vertical support rod, the rotating component and the horizontal support rod will become longer on the premise of meeting the needs of users, it will occupy more space, which will bring inconvenience whether it is used or stored; at the same time, the specifications of each component are too large, which will also increase the manufacturing cost; finally, due to the limitation of weight and specifications, the installation location of the device cannot be freely selected.

### SUMMARY

In order to solve the problems existing in the prior art, the present invention provides a device for helping a user to fix an oxygen hose, wherein a base is arranged, so that the device can be fixed on a plane such as a wall surface, thereby effectively utilizing space; the base is rotatably engaged with the bracket through the receiving part, and at least one clamp is arranged on the bracket, which is helpful for the user to adjust the position of the oxygen hose.

In order to achieve the above purpose, the technical solution of the present invention is as follows:
a device for helping a user to fix an oxygen hose includes a base, a bracket and at least one clamp;
wherein the base is detachably attached to a plane; the bracket is rotatably engaged on the base and has a rotation angle of 0 to 360 degrees relative to the base; and
wherein the base is provided with at least one receiving part, and the bracket comprises a fixed end, a movable end and at least one rotating feature; and
wherein, the receiving part receives the fixed end and forms a rotatable connection with the fixed end; and
wherein at least one clamp is arranged on the bracket far away from the fixed end; and
wherein the rotating feature is located between the movable end and the fixed end and facilitates a rotating movement of the movable end around at least one rotating axis of the fixed end, and wherein the rotating feature comprises an at least partially spherical surface, thereby facilitating a controlled change of a position of the movable end relative to the user by the user; and
wherein an edge of the base extends away from the base to form a base extension, and a hose hook for hanging the hose is formed on the base extension.

Through the solution disclosed by the present invention, the device for helping users to fix the oxygen hose can provide the base to be detachably attached to the plane, which can effectively utilize the space and meet the use requirements of some users; at the same time, the bracket is rotatably engaged with the base, at least one clamp is arranged on the bracket far away from the fixed end, and the movable end rotates around at least one rotation axis of the fixed end, so that the oxygen hose can be fixed on the bracket through the clamp on the bracket and can rotate with the bracket, and the oxygen hose can also be fixed on the hose hook on the base, thus being beneficial to the user to bundle up and fix the oxygen hose as required.

On the other hand, the present invention further provides: a device for helping a
user to fix an oxygen hose including
a base, a bracket and at least one clamp;
wherein the base is detachably attached to a plane, and the bracket is rotatably engaged on the base; and
wherein the base is provided with at least one receiving part, and the bracket comprises a fixed end, a movable end and at least one rotating feature; and
wherein, the receiving part receives the fixed end and forms a rotatable connection with the fixed end; and
wherein, the at least one clamp is arranged on the bracket far away from the fixed end; and
wherein the rotating feature is located between the movable end and the fixed end and facilitates a rotating movement of the movable end around at least one rotating axis of the fixed end, and wherein the rotating feature comprises an at least partially arc-shaped surface, thereby facilitating a controlled change of a position of the movable end relative to the user by the user.

Through the solution disclosed by the present invention, the device for helping users to fix the oxygen hose can provide the base to be detachably attached to the plane, which can effectively utilize the space and meet the use requirements of some users; at the same time, the bracket is rotatably engaged on the base, at least one clamp is arranged on the bracket far away from the fixed end, and the movable end rotates around at least one rotation axis of the fixed end, so that the oxygen hose can be fixed on the bracket through the clamp on the bracket and can rotate with the bracket, thereby being beneficial to users to bundle up and fix the oxygen hose as required.

Finally, the present invention further provides:
a hose hook of a ventilator, wherein the hose hook is detachably connected to a device for fixing an oxygen hose, the hose hook comprising a bottom seat, and two opposite ends of the bottom seat respectively extend to a same side of the bottom seat to form a first extension and a second extension; an end of the second extension extends towards the first extension to form a second resisting part;
the first extension and the second extension form at least one accommodating space, and anti-slip strip is arranged in the accommodating space, and the anti-slip strip is molded along the second resisting part by the first extension, wherein at least one opening is defined between the second resisting part and the first extension, and the opening defines a path for external objects to enter and leave the accommodating space.

Through the solution disclosed by the present invention, a user can fix an excessively long oxygen hose into the accommodating space, and the second resisting part at the end of the second extension can effectively prevent the oxygen hose from slipping off; the anti-slip strips arranged in the accommodating space can assist in fixing the oxygen hose.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the detailed description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

### BRIEF DESCRIPTION OF DRAWINGS

In order to explain the technical solution of this application more clearly, the drawings needed in the implementation will be briefly introduced below. Obviously, the drawings described below are only some implementations of this application. For those skilled in the art, other drawings can be obtained according to these drawings without creative work.

FIG. 1 is a schematic diagram of the overall structure of the present invention;

FIG. 2 is a schematic view of the present invention in an extended state;

FIG. 3 is a schematic view of the installation positions of the first clamp and the second clamp of the present invention;

FIG. 4 is a sectional view of the receiving part of the present invention;

FIG. 5 is a sectional view of the hinge structure of the connecting part and the rotating part of the present invention;

FIG. 6 is a schematic view of the hose hook of the present invention;

FIG. 7 is a schematic view of a hose hook with a third extension of the present invention;

FIG. 8 is a schematic diagram of the first clamp of the present invention;

FIG. 9 is a schematic view of the support member of the present invention;

FIG. 10 is a schematic diagram of the use method of the present invention;

FIG. 11 is a schematic view of the bunching hose of the present invention;

FIG. 12 is a schematic diagram of hanging a ventilator mask according to the present invention.

In the figures: 1, Base; 2, Bracket; 3, Receiving part; 4, Fixed end; 5, Movable end; 6, Limiting member; 7, First screw; 8, Knob; 9, Installation through hole; 10, First rotating arm; 11, Second rotating arm; 12, Connecting part; 13, Rotating part; 14, First clamp; 15, Support member; 16, Second screw; 17, Screw through hole; 18, Anti-slip rubber sleeve; 19, Second clamp; 20, Clamping part; 21, Bottom seat; 22, First extension; 23, Second extension; 24, Second resisting part; 25, Accommodating space; 26, Anti-slip strip; 27, Opening; 28, First resisting part; 29, Third extension; 30, Third resisting part; 31, Base extension; 32, Hose hook; 33, Flexible medium; 34, Ventilator; 35, Oxygen hose; 36, Ventilator mask.

### DESCRIPTION OF EMBODIMENTS

In describing the preferred embodiments, specific terminology will be resorted to for the sake of clarity. It is to be understood that each specific term includes all technical equivalents which operate in a similar manner to accomplish a similar purpose.

While various aspects and features of certain embodiments have been summarized above, the following detailed description illustrates a few exemplary embodiments in further detail to enable one skilled in the art to practice such embodiments. Reference will now be made in detail to embodiments of the inventive concept, examples of which are illustrated in the accompanying drawings. The accompanying drawings are not necessarily drawn to scale. The described examples are provided for illustrative purposes and are not intended to limit the scope of the invention. It should be understood, however, that persons having ordinary skill in the art may practice the inventive concept without these specific details.

It will be understood that, although the terms first, second, etc. may be used herein to describe various elements, these elements should not be limited by these terms. These terms are only used to distinguish one element from another. For example, a first attachment could be termed a second attachment, and, similarly, a second attachment could be termed a first attachment, without departing from the scope of the inventive concept.

It will be understood that when an element or layer is referred to as being “on,” “coupled to,” or “connected to” another element or layer, it can be directly on, directly coupled to or directly connected to the other element or layer, or intervening elements or layers may be present. In contrast, when an element is referred to as being “directly on,” “directly coupled to,” or “directly connected to” another element or layer, there are no intervening elements or layers present. Like numbers refer to like elements throughout. As used herein, the term “and/or” includes any and all combinations of one or more of the associated listed items.

As used in the description of the inventive concept and the appended claims, the singular forms “a,” “an,” and “the” are intended to include the plural forms as well, unless the context clearly indicates other.

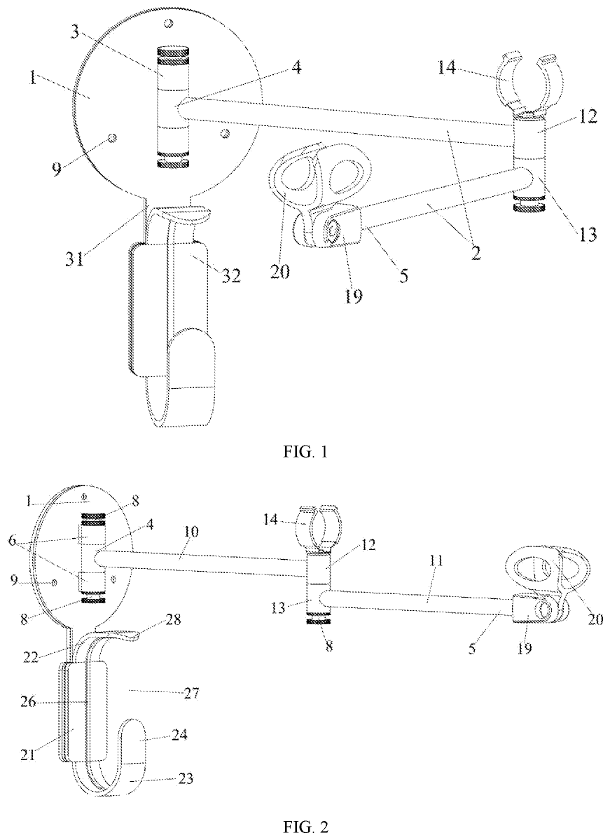

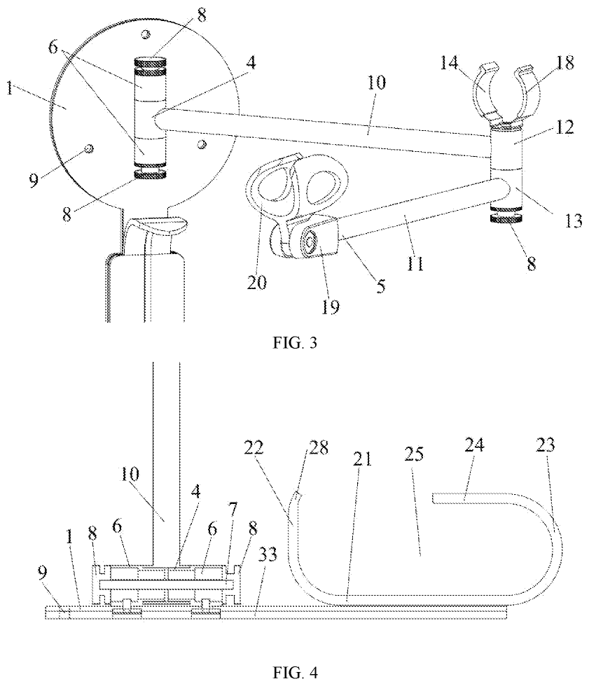

As shown in FIG. 1, a device for helping users to fix an oxygen hose includes a base 1, which is detachably attached to a plane, wherein the plane includes all object surfaces that meet the installation requirements of the device, including walls and cabinet sides, whether flat or curved; the base 1 is provided with a receiving part 3 which is rotatably connected with the bracket 2; at least one clamp is fixed on the bracket 2, and the clamp is used for fixing the hose of the ventilator, and the clamp is arranged far from the base 1; the position of the hose can be adjusted by rotating the bracket 2 around the receiving part 3, which is convenient for users to use.

In some embodiments, the base 1 is disc-shaped, and there are three installation through holes 9 for assisting the installation of equipment. Users can nail the base 1 to the wall or other surfaces with objects such as screws. The receiving part 3 comprises two limiting members 6 which are formed on one surface of the base 1 and are oppositely arranged, and the two limiting members 6 are hinged with the fixed end 4 of the bracket 2 together and have a damping structure, so that the bracket 2 is rotationally connected with the base 1; in another embodiment, the two limiting members 6 can also be fixed by screw connection.

In some embodiments, a first screw 7 and two knobs 8 are also used to fasten the two limiting members 6 and the bracket 2; when the knob 8 is loosened, the bracket 2 can rotate easily; when the knob 8 is tightened, the bracket 2 is fixed and keeps a fixed angle with the base 1; in another embodiment, the knob 8 is a flat cylinder with a concave middle part at the side and anti-slip threads at both ends.

In some embodiments, the bracket 2 is a straight rod, one end of the bracket 2 is rotationally connected with the base 1 as a fixed end 4, and the other end is used as a movable end 5, and a clamp for assisting in fixing the oxygen hose is installed; in another embodiment, the bracket 2 comprises a first rotating arm 10 and a second rotating arm 11, wherein one end of the first rotating arm 10 is rotationally connected with the base 1 as a fixed end 4, the other end is a connecting part 12, one end of the second rotating arm 11 is used as a movable end 5 to install a clamp, and the other end is used as a rotating part 13; the connecting part 12 is rotatably connected with the rotating part 13 and has a damping structure, and the second rotating arm 11 can make a circular motion of approximately 360 degrees around the hinge position of the connecting part 12 and the rotating part 13; preferably, both the connecting part 12 and the rotating part 13 have hollow structures, and the clamp can be fastened to the connecting part 12 by screws.

In another embodiment, the bracket 2 comprises a first rotating arm 10 and a second rotating arm 11, wherein one end of the first rotating arm 10 is rotationally connected with the base 1 as a fixed end 4, and the other end is a connecting part 12; one end of the second rotating arm 11 is used as a movable end 5 to install a second clamp 19, and the other end is used as a rotating part 13; the connecting part 12 is rotatably connected with the rotating part 13 and has a damping structure; and the second rotating arm 11 can make a circular motion of approximately 360 degrees around the hinge position of the connecting part 12 and the rotating part 13; both the connecting part 12 and the rotating part 13 have hollow structures, and the first clamp 14 can be fastened at the connecting part 12 by screws.

In some embodiments, the first clamp 14 installed at the connecting part 12 is provided with a screw through hole 17, a second screw 16 passes through the screw through hole 17 and the rotating shaft, and a knob 8 is screwed, so that the first clamp 14 is fastened at the connecting part 12 and can rotate. In another embodiment, a support member 15 is also installed between the first clamp 14 and the rotating shaft, and the support member 15 is partially inserted into the rotating shaft and separates the first clamp 14 from the rotating shaft, so that the friction suffered by the first clamp 14 when rotating is reduced.

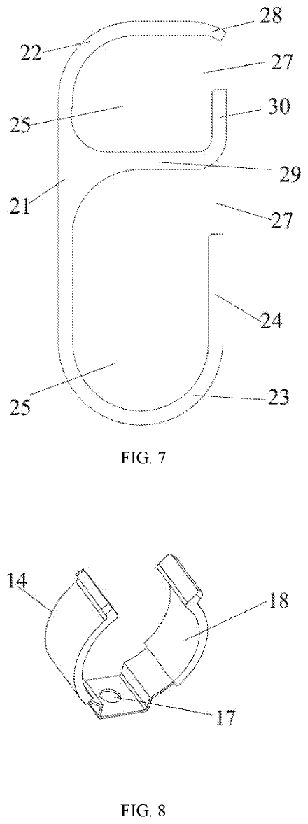

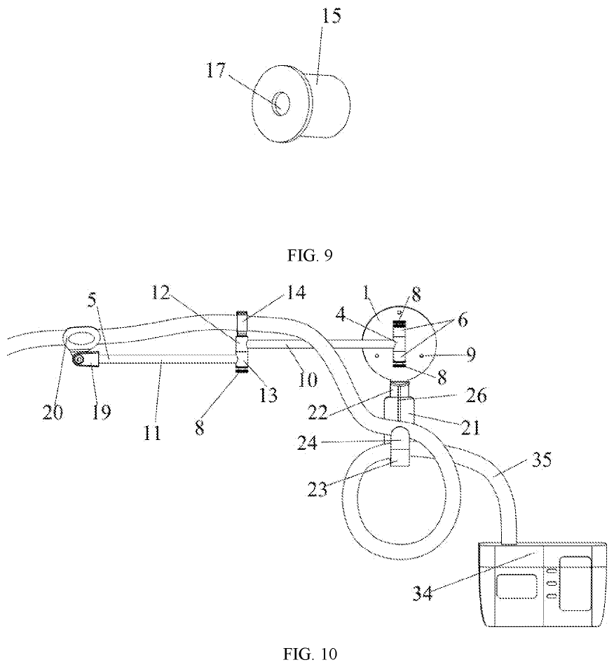

In some embodiments, as shown in FIG. 8, the first clamp 14 is also sheathed with an anti-slip rubber sleeve 18.

In some embodiments, the second clamp 19 is connected to the movable end 5 in a spiral way and includes a clamping part 20, and the rotation plane of the clamping part 20 is perpendicular to the rotation plane of the second rotating arm 11.

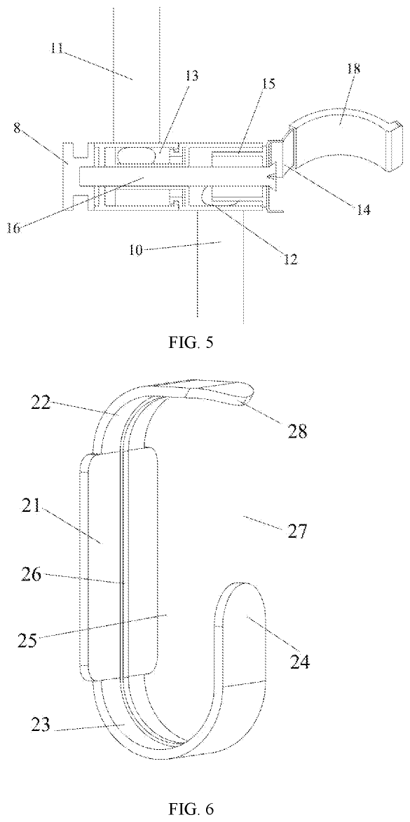

As shown in FIG. 6, the present invention also provides another solution, i.e., an oxygen hose hook:

The hose hook 32 is detachably connected to the device for fixing the oxygen hose, and the hose hook 32 comprises a bottom seat 21; and two ends of the base extend to one side of the bottom seat 21 to form a first extension 22 and a second extension 23 respectively; the end of the second extension 23 extends towards the first extension 22 to form a second resisting part 24, and the first extension 22 and the second extension 23 form at least one accommodating space 25, and anti-slip strip 26s are arranged in the accommodating space 25; and the anti-slip strip 26 is molded along the second resisting part 24 by the first extension 22, wherein at least one opening 27 is defined between the second resisting part 24 and the first extension 22, and the opening 27 defines the path for external objects to enter and leave the accommodating space 25.

In some embodiments, the first extension 22 further has a First resisting part 28 bent toward the second resisting part 24, further defining an opening 27 defined for the external object to pass through the first extension 22 and the second extension 23.

In some embodiments, the ends of the First resisting part 28 and the second resisting part 24 both have circular arc shapes, thereby facilitating the object to pass through the opening 27 and avoiding the accidental injury of the user.

In some embodiments, the lateral width of the first extension 22 and the second extension 23 is not greater than the lateral width of the bottom seat 21.

As shown in FIG. 7, in some embodiments, there is also a third extension 29 between the first extension 22 and the second extension 23, which divides the original accommodating space 25 into at least two smaller new accommodating spaces 25, and defines at least two opening 27s for objects to pass through with the first extension 22 and the second extension 23 respectively, and the anti-slip strips 26 extend through the third extension 29; in one embodiment, the end of the third extension 29 is also provided with a third resisting part 30, which extends towards the direction of the first extension 22; in another embodiment, the third resisting part 30 extends in the direction of the first extension 22 and the third extension 29.

In some embodiments, the anti-slip strip 26 is arranged along the bisector of the accommodating space 25, wherein the bisector is defined as a line that symmetrically bisects the oxygen hose hook along its length direction.

The present invention also provides another solution, namely, a device for helping a user to fix an oxygen hose:

The device comprises a base 1, a bracket 2 and at least one clamp, wherein the base 1 is detachably attached to a plane through an installation through holes 9; the bracket 2 is rotatably engaged on the base 1 and has a rotation angle of 0 to 360 degrees relative to the base 1; at least one receiving part 3 is arranged on the base 1, and the bracket 2 comprises a fixed end 4, a movable end 5 and at least one rotating feature; the receiving part 3 receives the fixed end 4 and forms a rotatable connection with the fixed end; at least one clamp is arranged on the bracket 2 far away from the fixed end 4; the rotating feature is located between the movable end 5 and the fixed end 4, and facilitates the rotating movement of the movable end 5 around at least one rotating axis of the fixed end 4, and wherein the rotating feature includes an at least partially spherical surface, thereby facilitating the controlled change of the position of the movable end 5 relative to the user by the user; the edge of the base 1 extends away from the base 1 to form a base extension 31, and a hose hook 32 for hanging the hose is formed on the base extension 31.

In some embodiments, the base 1 is disc-shaped, and there are three installation through holes 9 for assisting the installation of equipment. Users can nail the base 1 to the wall or other surfaces with objects such as screws. One side of the base 1 is also fixed with two limiting members 6 which are placed oppositely, and the two limiting members 6 are hinged with the fixed end 4 of the bracket 2 together and have a damping structure, so that the bracket 2 is rotationally connected with the base 1; in another embodiment, the two limiting members 6 can also be fixed by screw connection.

In some embodiments, a first screw 7 and two knobs 8 are also used to fasten the two limiting members 6 and the bracket 2, so that the bracket 2 and the base 1 can keep a fixed angle to meet the needs of users; in another embodiment, the knob 8 is a flat cylinder with a concave middle part at the side and anti-slip threads at both ends.

In some embodiments, the bracket 2 is a straight rod, one end of the bracket 2 is rotationally connected with the base 1 as a fixed end 4, and the other end is used as a movable end 5, and a clamp for assisting in fixing the oxygen hose is installed; in another embodiment, the bracket 2 comprises a first rotating arm 10 and a second rotating arm 11, wherein one end of the first rotating arm 10 is rotationally connected with the base 1 as a fixed end 4, the other end is a connecting part 12; one end of the second rotating arm 11 is used as a movable end 5 to install a clamp, and the other end is used as a rotating part 13, the connecting part 12 is rotatably connected with the rotating part 13 and has a damping structure, and the second rotating arm 11 can make a circular motion of approximately 360 degrees around the hinge position of the connecting part 12 and the rotating part 13; preferably, both the connecting part 12 and the rotating part 13 have hollow structures, and the clamp can be fastened to the connecting part 12 by the second screw 16.

In another embodiment, the bracket 2 comprises a first rotating arm 10 and a second rotating arm 11, wherein one end of the first rotating arm 10 is rotationally connected with the base 1 as a fixed end 4, and the other end is a connecting part 12; one end of the second rotating arm 11 is used as a movable end 5 to install a second clamp 19, and the other end is used as a rotating part 13; the connecting part 12 is rotatably connected with the rotating part 13 and has a damping structure; and the second rotating arm 11 can make a circular motion of approximately 360 degrees around the hinge position of the connecting part 12 and the rotating part 13. Both the connecting part 12 and the rotating part 13 have hollow structures, and the first clamp 14 can be fastened to the connecting part 12 by the second screw 16.

In some embodiments, the first clamp 14 installed at the connecting part 12 is provided with a screw through hole 17, the second screw 16 passes through the screw through hole 17 and the rotating shaft, and a knob 8 is screwed, so that the first clamp 14 is fastened at the connecting part 12 and can rotate. In another embodiment, a support member 15 is also installed between the first clamp 14 and the rotating shaft, and the support member 15 is partially inserted into the rotating shaft and separates the first clamp 14 from the rotating shaft, so that the friction suffered by the first clamp 14 when rotating is reduced.

In some embodiments, the second clamp 19 is connected to the movable end 5 in a spiral way and includes a clamping part 20, and the rotation plane of the clamping part 20 is perpendicular to the rotation plane of the second rotating arm 11.

In some embodiments, a hose hook 32 is attached to the extension of the base 1, and the hose hook 32 is on the same side as the bracket 2; the hose hook 32 comprises a bottom seat 21, and two ends of the bottom seat 21 respectively extend to one side of the bottom seat 21 to form a first extension 22 and a second extension 23; the end of the second extension 23 extends towards the first extension 22 to form a second resisting part 24, the first extension 22 and the second extension 23 form at least one accommodating space 25, anti-slip strip 26 is arranged in the accommodating space 25, and the anti-slip strip 26 is molded along the second resisting part 24 by the first extension 22, wherein at least one opening 27 is defined between the second resisting part 24 and the first extension 22, and the opening 27 defines the path for external objects to enter and leave the accommodating space 25; in another embodiment, the hose hook 32 is molded on the base extension 31.

In some embodiments, the first extension 22 further has a first resisting part 28 bent toward the second resisting part 24, thereby further limiting the passage of external objects through the opening 27 defined by the first extension 22 and the second extension 23.

In some embodiments, the ends of the first resisting part 28 and the second resisting part 24 both have circular arc shapes, thereby facilitating the object to pass through the opening 27 and avoiding the accidental injury of the user.

In some embodiments, the lateral width of the first extension 22 and the second extension 23 is not greater than the lateral width of the bottom seat 21.

In some embodiments, there is also a third extension 29 between the first extension 22 and the second extension 23, which divides the original accommodating space 25 into at least two smaller new accommodating space 25s, and defines at least two openings 27 for objects to pass through with the first extension 22 and the second extension 23 respectively, and the anti-slip strip 26 extends through the third extension 29; in one embodiment, the end of the third extension 29 is also provided with a third resisting part 30, which extends towards the direction of the first extension 22; in another embodiment, the third resisting part 30 extends in the direction of the first extension 22 and the third extension 29.

In some embodiments, the anti-slip strip 26 is arranged along the bisector of the accommodating space 25, wherein the bisector is defined as a line that symmetrically bisects the oxygen hose hook along its length direction.

In some embodiments, the use method of the present invention is as follows:
Step 1, installing the product on the wall by using screws to pass through the installation through holes 9;
Step 2, rotating the bracket 2 of the product, so that the first rotating arm 10 and the second rotating arm 11 rotate to proper positions, and tightening the knob 8 of the receiving part 3 and the knob 8 screwed with the second screw 16 to fix the positions of the first rotating arm 10 and the second rotating arm 11;
Step 3, putting the oxygen hose 35 into the accommodating space 25;
Step 4, fixing the oxygen hose 35 by the first clamp 14 and the second clamp 19 respectively.

In some embodiments, as shown in FIG. 10, the use method of the device for helping users to fix the oxygen hose provided by the solution of the present invention is as follows: rotating the bracket 2 to a proper position, and tightening the knobs 8 of the receiving part 3 and the rotating part 13 to keep the bracket 2 in an extended state; putting the oxygen hose 35 into the accommodating space 25 of the hose hook 32, and after binding the excess part of the oxygen hose 35, using the first clamp 14 and the second clamp 19 to clamp the oxygen hose 35.

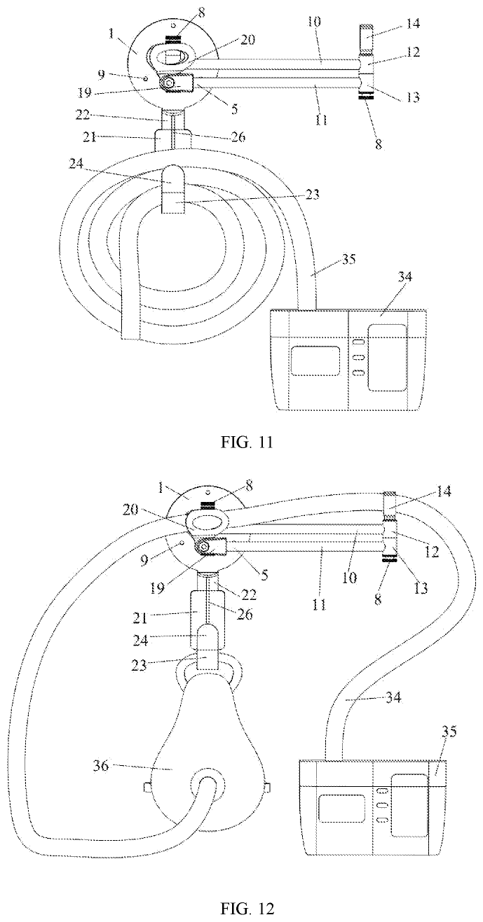

In other embodiments, the device provided by the solution of the present invention for helping users to fix the oxygen hose has another use method, as shown in FIG. 11, when the user does not need to use the ventilator 34 temporarily, the bracket 2 is folded; at the same time, the oxygen hose 35 is coiled up and put into the accommodating space 25 of the hose hook 32.

In still other embodiments, the device for helping users to fix the oxygen hose provided by the solution of the present invention has another use method, as shown in FIG. 12, when the user does not need to use the ventilator 34 temporarily, the bracket 2 is folded; the hose hook 32 can be used to hang ventilator masks 36.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

The invention has now been described in detail for the purposes of clarity and understanding. However, those skilled in the art will appreciate that certain changes and modifications may be practiced within the scope of the appended claims.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

What is claimed is:

1. A device for helping a user to fix an oxygen hose, comprising a base, a bracket and at least one clamp; and
wherein said base is detachably attached to a plane; and said bracket is rotatably engaged on said base and has a rotation angle of 0 to 360 degrees relative to said base; and
wherein said base is provided with at least one receiving part, and said bracket comprises a fixed end, a movable end and at least one rotating feature; and
wherein, said receiving part receives said fixed end and forms a rotatable connection with said fixed end; and
wherein at least one clamp is arranged on said bracket far away from said fixed end; and
wherein said rotating feature is located between said movable end and said fixed end and facilitates a rotating movement of said movable end around at least one rotating axis of said fixed end, and wherein said rotating feature comprises an at least partially spherical surface, thereby facilitating a controlled change of a position of said movable end relative to the user by the user; and
wherein an edge of said base extends away from said base to form a base extension, and a hose hook for hanging the hose is formed on said base extension.

2. The device for helping a user to fix an oxygen hose according to claim 1, wherein said receiving part comprises two limiting members fixed on said base, which are oppositely arranged, hinged with said fixed end together and have a damping structure; and
wherein, said receiving part further comprises a first screw, which passes through said fixed end and said two limiting members, and two ends of said first screw are respectively connected with two knobs in a threaded way.

3. The device for helping a user to fix an oxygen hose according to claim 1, wherein said base is in a sheet shape, and said base is provided with an installation through hole, thereby assisting said base to be detachably attached to the plane.

4. The device for helping a user to fix an oxygen hose according to claim 1, wherein said bracket comprises a first rotating arm and a second rotating arm; and one end of said first rotating arm is said fixed end, and the other end of said second rotating arm is provided with a connecting part; and the other end of said second rotating arm is provided with a rotating part, wherein said connecting part forms a rotatable connection with said rotating part, and both said connecting part and said rotating part have hollow structures.

5. The device for helping a user to fix an oxygen hose according to claim 4, further comprising a first clamp, a support member and a second screw, wherein both said first clamp and said support member have a screw through holes for said second screw to pass through and said support member is installed between said connecting part and said first clamp.

6. The device for helping a user to fix an oxygen hose according to claim 5, wherein said first clamp is in an open annular shape, and said screw through hole is arranged in a middle section of said first clamp and protrudes outward along a radial direction of said first clamp; and
wherein two ends of said first clamp are provided with anti-slip rubber sleeves, and the ends of said first clamp are gradually away from each other.

7. The device for helping a user to fix an oxygen hose according to claim 1, further comprising a flexible medium, wherein said flexible medium is stuck on a side of said base which is not connected with said bracket.

8. The device for helping a user to fix an oxygen hose according to claim 1, wherein said hose hook is stuck on said base extension; and
wherein, said hose hook comprises a bottom seat, and two opposite ends of said bottom seat respectively extend to a same side of said bottom seat to form a first extension and a second extension; and an end of said second extension extends towards said first extension to form a second resisting part; and
said first extension and said second extension form at least one accommodating space, and anti-slip strip is arranged in said accommodating space and said anti-slip strip is molded along said second resisting part by said first extension, wherein at least one opening is defined between said second resisting part and said first extension, and said opening defines a path for external objects to enter and leave said accommodating space.

9. The device for helping a user to fix an oxygen hose according to claim 8, wherein said first extension further has a first resisting part bent toward said second resisting part; and
wherein, the ends of said first resisting part and said second resisting part both have arc shapes; and
wherein the lateral widths of said first extension and said second extension are not greater than the lateral width of said bottom seat.

10. The device for helping a user to fix an oxygen hose according to claim 8, further comprising a third extension arranged between said first extension and said second extension, and a third resisting part is further arranged at an end of said third extension; and wherein, said anti-slip strip is arranged along a bisector of said accommodating space.

11. A device for helping a user to fix an oxygen hose, comprising a base, a bracket and at least one clamp; and
wherein said base is detachably attached to a plane, and said bracket is rotatably engaged on said base; and
wherein said base is provided with at least one receiving part, and said bracket comprises a fixed end, a movable end and at least one rotating feature; and
wherein, said receiving part receives said fixed end and forms a rotatable connection with said fixed end; and
wherein, said at least one clamp is arranged on said bracket far away from said fixed end; and
wherein said rotating feature is located between said movable end and said fixed end and facilitates a rotating movement of said movable end around at least one rotating axis of said fixed end, and wherein said rotating feature comprises an at least partially arc-shaped surface, thereby facilitating a controlled change of a position of said movable end relative to the user by the user.

12. The device for helping a user to fix an oxygen hose according to claim 11, wherein said receiving part comprises two limiting members fixed on said base, and said limiting members are oppositely arranged, hinged with said fixed end together and have a damping structure; and wherein, said receiving part further comprises:
a first screw, which passes through said fixed end and said two limiting members, wherein two ends of said first screw are respectively connected with two knobs by threads; and said knob is a flat cylinder with a concave middle part on a side and anti-slip threads on both ends.

13. The device for helping a user to fix an oxygen hose according to claim 11, wherein said base is in a sheet shape and is provided with an installation through hole, thereby assisting said base to be detachably attached to the plane.

14. The device for helping a user to fix an oxygen hose according to claim 11, wherein said bracket comprises a first rotating arm and a second rotating arm; and one end of said first rotating arm is said fixed end, and the other end of said second rotating arm is provided with a connecting part; and the other end of said second rotating arm is provided with a rotating part, wherein said connecting part forms a rotatable connection with said rotating part, and both said connecting part and said rotating part have hollow structures.

15. The device for helping a user to fix an oxygen hose according to claim 14, further comprising a first clamp, a support member and a second screw, wherein both said first clamp and said support member have a screw through holes for said second screw to pass through and said support member is installed between said connecting part and said first clamp.

16. The device for helping a user to fix an oxygen hose according to claim 15, wherein said first clamp is in an open annular shape, and said screw through hole is arranged in a middle section of said first clamp and protrudes outward along a radial direction of said first clamp; and
wherein two ends of said first clamp are provided with anti-slip rubber sleeves, and the ends of said first clamp are gradually away from each other.

17. The device for helping a user to fix an oxygen hose according to claim 15, wherein said movable end of said second rotating arm is connected with a second clamp in a screw thread and said second clamp comprises a clamping part which rotates around said movable end, and a rotation plane of said clamping part is perpendicular to a rotation plane of said second rotating arm.

18. A hose hook of a ventilator, wherein said hose hook is detachably connected to a device for fixing an oxygen hose, said hose hook comprising a bottom seat, and two opposite ends of said bottom seat respectively extend to a same side of said bottom seat to form a first extension and a second extension; and an end of said second extension extends towards said first extension to form a second resisting part; and
said first extension and said second extension form at least one accommodating space, and anti-slip strip is arranged in said accommodating space and said anti-slip strip is molded along said second resisting part by said first extension, wherein at least one opening is defined between said second resisting part and said first extension, and said opening defines a path for external objects to enter and leave said accommodating space.

19. The hose hook according to claim 18, wherein said first extension further has a first resisting part bent to said second resisting part; and
wherein, the ends of said first resisting part and said second resisting part both have arc shapes; and
wherein the lateral widths of said first extension and said second extension are not greater than the lateral width of said bottom seat.

20. The hose hook according to claim 18, further comprising a third extension arranged between said first extension and said second extension, and a third resisting part is further arranged at an end of said third extension; and
wherein, said anti-slip strip is arranged along a bisector of said accommodating space.
